(19) United States
(12) Patent Application Publication
(10) Pub. No.: US 2006/0089278 A1
(43) Pub. Date: Apr. 27, 2006

(54) Liquid cleansing compositions

(75) Inventor: Niraj Dhansukhlal Mistry, Mumbai (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/255,378

(22) Filed: Oct. 21, 2005

(30) Foreign Application Priority Data
Oct. 26, 2004 (IN) 1144/MUM/2004

(51) Int. Cl. A61K 8/00

(52) U.S. Cl. 510/130

(57) ABSTRACT

A liquid cleansing composition comprising 5% to 25% by weight of soap, 5% to 20% by weight humectants, balance water; wherein the soap comprises 0.05% to 3% by weight of the composition, salt of unsaturated fatty acid.

The present invention relates to cleansing compositions in liquid or gel form. More particularly, it relates to transparent liquid soap compositions which can be prepared at low cost. The invention also relates to a process for preparing the cleansing compositions.

Liquid soaps compositions, particularly transparent soaps, have aesthetic appeal. These are often used for specialised applications like hand wash and face wash, and are particularly preferred for out-of-home applications such as during travel, at hotels and restaurants, where people are very conscious of hygiene, and where there is possibility of contamination at the wash place due to the large number of people using the soap.

Liquid soaps are usually alkali metal salts of fatty acids which are saturated. Among the alkali metals, potassium is preferred over sodium, as potassium fatty acid soaps are more soluble in water, thereby maintaining the liquid state in the formulation. However potassium salts are more expensive, and there is a constant need to reduce cost by using more inexpensive salts like sodium salts of fatty acids. Of late, there is also a trend to incorporate non-soap detergents (NSD) that provide improved solubility in water, but these materials tend to be more expensive. Moreover, some of the synthetic detergents have certain consumer-perceived negatives, such as skin feel and ease of rinsing.

There is a need felt in the art to incorporate high level of total fatty matter to ensure better cleaning, at the same time maintaining the fluid state and improving the transparency of the composition. It has been found by way of the present invention that a small amount of a soap of unsaturated fatty acids, when present in the composition along with selected humectants, ensures fluidity in the composition, and allows for a wider formulation window, such as higher use of sodium soaps and lower use of non-soap detergents. It also allows for the use of humectants, which otherwise result in loss of product clarity.

U.S. Pat. No. 4,310,432 (Lever Brothers Company, 1982) claims an aqueous liquid soap solution containing from 20% to 45% by weight of a sodium soap consisting essentially of a mixture of (A) at least one sodium soap of C₈ to C₁₄ saturated fatty acid, and (B) at least one sodium soap of fatty acids selected from the group consisting essentially of C₁₆ to C₂₂ mono and di unsaturated fatty acids and mixtures thereof, the ratio of A to B being in the range of from 4:1 to 1:4. Examples of component B as given in U.S. Pat. No. 4,310,432 are pure or technical grades of commercially available oleic acid, ricinoleic acid, fatty acids derived from castor oil and from rapeseed oil, and mixtures thereof.

The invention has been largely demonstrated at amounts of the unsaturated fatty acid soaps as a percentage of total fatty acid soaps in the range of 20% to 85%. The use of such high amounts of unsaturated fatty acids soaps in the liquid cleansing compositions leads to problems of stability which are corrected by inclusion of stabilising agents which add to the cost. Moreover, generation of lather which is an important consumer attribute is also affected.

The present inventors have now found that when surprisingly low amounts of unsaturated fatty acid soaps are incorporated in the liquid/gel cleansing compositions comprising humectants, the compositions are highly fluid, provide excellent cleaning performance, the saturated fatty acid soaps could be of almost any molecular weight, and it is possible to include high levels of sodium soaps.

It is thus an object of the present invention to be able to provide for a liquid/gel cleansing composition with good transparency, and therefore high consumer appeal.

It is a further object of the present invention to be able to provide for a transparent liquid/gel cleansing composition which comprises higher amount of soaps prepared from higher molecular weight fatty acids and sodium soaps which are more readily available and are of lower cost, and therefore are more economical to prepare.

It is a further object of the present invention to be able to provide for a transparent liquid/gel cleansing composition which is prepared using very low amounts of unsaturated fatty acids, thereby giving enhanced stability of the soap composition.

It is yet another object of the invention to be able to provide for a liquid/gel cleansing composition which retains its transparency using very low or no amount of synthetic surfactants.

According to a first aspect of the invention, there is provided for a liquid cleansing composition comprising:
5% to 25% by weight of soap,
5% to 20% by weight humectants,
balance water,
   wherein the composition comprises 0.05% to 3% by weight of the composition of a salt of unsaturated fatty acid.

According to a further aspect of the invention, there is provided a process for preparing a liquid cleansing composition of the invention comprising:
5% to 25% by weight of soap,
5% to 20% by weight humectants,
water,
   wherein the composition comprises 0.05% to 3% by weight of the composition, salt of unsaturated fatty acid, which process comprises the steps of:
(i) saponifying an oil with an alkali,
(ii) diluting with desired amount of water, and
(iii) mixing the humectants and optionally other additives.

It is particularly preferred that the humectant is chosen from sorbitol or propylene glycols.

The particularly preferred oil is coconut oil or palm kernel oil.

Throughout the specification, all parts are by weight unless otherwise specified.

By the word “transparent” is meant that the soap composition is capable of transmitting light therethrough.

By the word “liquid” is meant liquids in the fluid state and in the gel state.

The present invention relates to liquid cleansing compositions comprising soap, humectants and water wherein 0.05% to 3% by weight of the composition is a salt of unsaturated fatty acid.

The soap of the present invention are salts of fatty acids. Suitable fatty acids are the C₈-C₂₂ fatty acids. Fatty acids particularly suitable for the invention include lauric acid, myristic acid, palmitic acid and stearic acid. These can also be obtained from plant and/or animal sources, for example tallow fatty acids, palm fatty acids, palm kernel fatty acids etc.

The term soap refers to the salts of these fatty acids.

Suitable cations include sodium, potassium, zinc, magnesium, alkyl ammonium and aluminium. Alkali metal cations, viz. potassium and sodium are especially preferred. The invention is especially suitable for incorporation of sodium soaps along with potassium soaps. Sodium soaps at up to 20%, 40%, 60%, 80% or 100% of the total soap is possible.

It is more preferred to saponify oils or their mixtures instead of neutralising the fatty acids. Suitable oils for the invention include tallow, tallow stearines, palm oil, palm stearines, soya bean oil, fish oil, rice bran oil, sunflower oil, coconut oil, babassu oil and palm kernel oil. Especially preferred oils are coconut oil and palm kernel oil.

The fatty acid soaps can also be prepared by a synthetic process, e.g. by the oxidation of petroleum, or by the hydrogenation of carbon monoxide by the Fischer-Tropsch process.

The soap content of the liquid cleansing composition is from 5% to 25%, more preferably from 10% to 22%, and most preferably from 18% to 22%.

The liquid cleansing composition of the invention comprises salt of unsaturated fatty acids, present in an amount of 0.05% to 2.75%, more preferably 0.05% to 2.5%, more preferably 1.0% to 2.5% by weight of the composition.

Humectants are essential as per the composition of the invention, and include polyhydric alcohols. Polyhydric alcohols suitable for the invention include polyethylene glycol, propylene glycol, glycerol and sorbitol. Especially preferred humectants are propylene glycol and/or sorbitol.

The polyhydric alcohol is present in an amount of from 5% to 20%, more preferably from 12% to 18% by weight of the composition.

Optional ingredients include non-soap detergent actives, which can be optionally added to the composition of the invention.

They may be suitably added after the soap is mixed with water and the humectants. Non-soap detergent actives may be chosen from anionic, cationic, zwitterionic, amphoteric surfactants or their mixtures thereof. When present, the preferred non-soap detergent active is chosen from anionic or non-ionic type.

Preferably the amount of the non-soap detergent active does not exceed 20%.

A suitable class of anionic surfactants are water-soluble salts of organic sulphuric acid mono-esters and sulphonic acids having in the molecular structure a branched or straight chain alkyl group containing 8-22 C atoms or an alkylaryl group containing 6-20 C atoms in the alkyl part.

Examples of such anionic surfactants are water soluble salts of:
long chain (i.e. 8-22 C-atom) alcohol sulphates (hereinafter referred to as PAS), especially those obtained by sulphating the fatty alcohols produced from tallow or coconut oil or the synthetic alcohols derived from petroleum;
alkylbenzene-sulphonates, such as those in which the alkyl group contains from 6 to 20 carbon atoms;
secondary alkanesulphonates.

Also suitable are the salts of:
alkylglyceryl ether sulphates, especially of the ethers of fatty alcohols derived from tallow and coconut oil;
fatty acid monoglyceride sulphates;
sulphates of ethoxylated aliphatic alcohols containing 1-12 ethyleneoxy groups;
alkylphenol ethylenoxy-ether sulphates with from 1 to 8 ethyleneoxy units per molecule and in which the alkyl groups contain from 4 to 14 carbon atoms;
the reaction product of fatty acids esterified with isethionic acid and neutralised with alkali.

Suitable nonionic detergent active compounds can be broadly described as compounds produced by the condensation of alkylene oxide groups, which are hydrophilic in nature, with an organic hydrophobic compound which may be aliphatic or alkyl aromatic in nature. The length of the hydrophilic or polyoxyalkylene radical which is condensed with any particular hydrophobic group can be readily adjusted to yield a water-soluble compound having the desired degree of balance between hydrophilic and hydrophobic elements. Other nonionic surfactants that can be employed include alkylpolyglucosides.

Particular examples include the condensation product of aliphatic alcohols having from 8 to 22 carbon atoms in either straight or branched chain configuration with ethylene oxide, such as a coconut oil ethylene oxide condensate having from 2 to 15 moles of ethylene oxide per mole of coconut alcohol; condensates of alkylphenols whose alkyl group contains from 6 to 12 carbon atoms with 5 to 25 moles of ethylene oxide per mole of alkylphenol; condensates of the reaction product of ethylenediamine and propylene oxide with ethylene oxide, the condensate containing from 40% to 80% of polyoxyethylene radicals by weight and having a molecular weight of from 5,000 to 11,000; tertiary amine oxides of structure R₃NO, where one group R is an alkyl group of 8 to 18 carbon atoms and the others are each methyl, ethyl or hydroxyethyl groups, for instance dimethyldodecylamine oxide; tertiary phosphine oxides of structure R₃PO, where one group R is an alkyl group of from 10 to 18 carbon atoms, and the others are each alkyl or hydroxyalkyl groups of 1 to 3 carbon atoms, for instance dimethyldodecylphosphine oxide; and dialkyl sulphoxides of structure R₂SO where the group R is an alkyl group of from 10 to 18 carbon atoms and the other is methyl or ethyl, for instance methyltetradecyl sulphoxide; fatty acid alkylolamides; alkylene oxide condensates of fatty acid alkylolamides and alkyl mercaptans.

Further examples of suitable detergent-active compounds are compounds commonly used as surface-active agents given in the well-known textbooks “Surface Active Agents”, Volume I by Schwartz and Perry and “Surface Active Agents and Detergents”, Volume II by Schwartz, Perry and Berch.

Salts are optionally added to the soap composition of the invention. Suitable salts include sodium and potassium salts. Sodium chloride is an especially preferred salt and is preferably used in an amount of from 0.1% to 2%.

Other optional ingredients like anti-oxidants, perfumes, polymers, thickening agents, chelating agents, colourants, deodorants, dyes, emollients, moisturisers, enzymes, foam boosters, germicides, anti-microbials, lathering agents, pearlescers, skin conditioners, solvents, stabilisers, superfatting agents, sunscreens etc. may be added in suitable amounts in the process of the invention, provided the transparency of the soap is retained. Preferably, the ingredients are added after the essential ingredients are mixed in the composition.

Another aspect of the invention provides for a process for preparing a liquid cleansing composition comprising 5% to 25% soap, 5% to 20% humectants, and balance water wherein the composition comprises 0.05% to 3% by weight of the composition, salt of unsaturated fatty acid which process comprises the steps of:
(i) saponifying an oil with an alkali;
(ii) diluting with desired amount of water; and
(iii) mixing the humectants and optionally other additives.

The process of the invention which comprises saponifying oil is preferred over a process which comprises neutralising a fatty acid mix. The preferred oils as per the invention include coconut oil or palm kernel oil. It has been found that saponifying the oils with an alkali produces the fatty acid soaps in the desired concentrations, along with the side product viz. glycerol present in the liquid mixture. Further dilution with water and addition of humectants to the liquid mixture, thus prepared, produces the liquid cleansing composition of the invention.

The process of the invention is preferable over the route that involves neutralisation of fatty acid mix, followed by dilution with water and addition of humectants.

Neutralisation of fatty acid involves starting with the raw material viz. fatty acid. Preparation of fatty acid as a raw material involves the following additional process steps as compared to the process step used in preparing the soap of the invention: (i) Fat splitting; (ii) Distillation for purification of the fatty acid; (iii) Drying of the fatty acid to remove water; (iv) Hydrogenation of the fatty acid; and (v) separation of the glycerol from the saponified mix. All these extra process steps involve additional cost which would not be incurred by the preferred process of the invention.

The invention will be further described by the following illustrative non-limiting examples. All parts therein are by weight unless otherwise specified.

### EXAMPLES

### Comparative Examples A to D

Compositions of the prior art were prepared by neutralising a mixture of saturated fatty acids, viz. lauric acid and myristic acid in a weight ratio of 3:2 with alkali, followed by addition of water and humectants to prepare compositions as shown in Table 1. The visual appearance of the compositions are also summarized.

TABLE 1

| | Comp. | Comp. | Comp. | Comp. |
|---|---|---|---|---|
| Ingredient | Example A | Example B | Example C | Example D |
| Soap (wt %) | 22 | 22 | 22 | 22 |
| Alkali used | KOH | NaOH | KOH | NaOH |
| Glycerine | 10 | 10 | — | — |
| Propylene | — | — | 15 | 15 |
| glycol | | | | |
| Polyethylene | 6 | 6 | — | — |
| glycol | | | | |
| Water | To 100 | To 100 | To 100 | To 100 |
| Product state | Liquid | Solid | Liquid | Solid |
| Product clarity | Clear | Opaque | Clear | Opaque |

Comparative examples A to D are examples of cleansing compositions of the prior art which do not comprise salts of unsaturated fatty acids. The compositions require the use of potassium soaps for enabling the product to be in the liquid state and clear. When sodium soaps are used, the corresponding compositions are solid and opaque.

### Examples 1 to 3

Compositions as per the inventions were prepared by saponifying palm kernel oil (PKO) with sodium hydroxide. PKO used has the following fatty acids as shown in Table 2.

TABLE 2

| Fatty acid | Carbon chain length | Wt % |
|---|---|---|
| Lauric acid | C 12 | 54.2 |
| Myristic acid | C 14 | 17.0 |
| Palmitic acid | C 16 | 12.0 |
| Stearic acid | C 18 | 3.9 |
| Oleic acid | C 18:1 | 9.9 |
| Linoleic acid | C18:2 | 1.2 |

Compositions as shown in Table 3 were prepared by mixing the product obtained from saponification of the PKO, with the humectants indicated and the composition was made up to 100% with water. The visual appearance of the compositions is also summarized in Table 3.

TABLE 3

| Ingredient | Example 1 | Example 2 | Example 3 |
|---|---|---|---|
| Soap (wt %) | 23.4 | 23.4 | 22 |
| % salt of unsaturated | 2.6 | 2.6 | 2.44 |
| fatty acid | | | |
| Alkali used | KOH | KOH | NaOH |
| Propylene glycol | 15 | — | 15 |
| Sorbitol | — | 15 | — |
| Water | To 100 | To 100 | To 100 |
| Product state | Liquid | Liquid | Liquid |
| Product clarity | Clear | Clear | Clear |

The data in Table 3 indicates that compositions which include salt of unsaturated fatty acid provide for cleansing compositions in the liquid state which are visually clear, even when the salts are sodium salts.

### Examples 4 to 9

Cleansing compositions of the invention were prepared as per Examples 1 to 3 which included non-soap detergent actives in various amounts and the details are shown in Table 4. The visual appearance of the product is also summarized.

TABLE 4

| Ingredient | Ex 4 | Ex 5 | Ex 6 | Ex 7 | Ex 8 | Ex 9 |
|---|---|---|---|---|---|---|
| Soap (wt %) | 22.0 | 22.0 | 22.0 | 22.0 | 17.6 | 17.6 |
| % salt of | 2.44 | 2.44 | 2.44 | 2.44 | 1.95 | 1.95 |
| unsaturated | | | | | | |
| fatty acid | | | | | | |
| Alkali used | NaOH | NaOH | NaOH | NaOH | NaOH | NaOH |
| Propylene | — | — | — | — | — | — |
| glycol | | | | | | |
| Sorbitol | 15 | 15 | 15 | 10 | 15 | 15 |
| Non-soap | SLES | SLES | LAO | LAO | SLES | LAO |
| detergent(NSD) | | | | | | |
| Wt % | 0.75 | 1.5 | 0.75 | 1.0 | 1.0 | 1.0 |
| Water | To 100 | To 100 | To 100 | To 100 | To 100 | To 100 |
| Product state | Gel | Soft Gel | Liquid | Liquid | Gel | Liquid |
| Product clarity | Clear | Clear | Clear | Clear | Clear | Clear |

SLES: Sodium Lauryl ethoxy sulphate.
LAO: Lauryl amine oxide.

The data in Table 4 indicates that cleansing compositions with sodium soaps which comprise salt of unsaturated fatty acids and small amount of non-soap detergent active provide for compositions in the liquid/gel state and which are visually clear.

1. A liquid cleansing composition comprising:
5% to 25% by weight of soap,
5% to 20% by weight humectants,
water;
wherein the soap comprises 0.05% to 3% by weight of the composition, salt of unsaturated fatty acid.

2. The liquid cleansing composition as claimed in claim 1 wherein the soap comprises alkali metal salts of C₈ to C₂₀ fatty acids.

3. The liquid cleansing composition as claimed in claim 1 wherein the soap is present in 10% to 22% by weight of the composition.

4. The liquid cleansing composition as claimed in claim 3 wherein the soap is present in 18% to 22% by weight of the composition.

5. The liquid cleansing composition as claimed in claim 1 wherein the salt of unsaturated fatty acid is present in an amount of 1.0% to 2.5% by weight of the composition.

6. The liquid cleansing composition as claimed in claim 1 wherein the humectant is selected from polyhydric alcohols including glycerol, sorbitol, polyethylene glycols, propylene glycols or mixtures thereof.

7. The liquid cleansing composition as claimed in claim 6 wherein the humectant is selected from sorbitol, propylene glycols or mixtures thereof.

8. The liquid cleansing composition as claimed in claim 1 wherein the humectant is present in 12% to 18% by weight of the composition.

9. The liquid cleansing composition as claimed in claim 1 comprising not more than 20% non-soap detergent active by weight of the composition.

10. The liquid cleansing composition as claimed in claim 9 comprising not more than 5% non-soap detergent active by weight of the composition.

11. The liquid cleansing composition as claimed in claim 1 wherein the composition is transparent.

12. A process for preparing the liquid cleansing composition as claimed in claim 1 comprising:
(i) saponifying an oil with a alkali
(ii) diluting with desired amount of water; and
(iii) mixing the humectants and optionally other additives.

13. The process as claimed in claim 12 wherein the oil is coconut oil or palm kernel oil.
